(19) United States
(12) Patent Application Publication
(10) Pub. No.: US 2017/0182277 A1
(43) Pub. Date: Jun. 29, 2017

(54) SYSTEM AND METHOD FOR ADDRESSING HYPOXEMIA

(71) Applicant: Ethicon Endo-Surgery, Inc., Cincinnati, OH (US)

(72) Inventors: Paul J. Niklewski, Cincinnati, OH (US); James F. Martin, Lebanon, OH (US)

(21) Appl. No.: 14/982,235

(22) Filed: Dec. 29, 2015

(51) Int. Cl. A61M 16/10; A61M 5/172; A61M 16/06; A61M 5/142; A61M 16/00; A61B 5/1455

(52) U.S. Cl. CPC A61M 16/1005; A61M 16/0051; A61B 5/14551; A61M 16/0666; A61M 5/142; A61M 5/1723; A61M 2230/205; A61M 2202/0208; A61M 2230/005; A61M 2230/432; A61M 2230/42

(57) ABSTRACT

A patient monitoring system is configured to monitor oxygen saturation and/or oxygenation of a patient's blood. The system is configured to re-oxygenate the patient in response to a determination that the patient's oxygen saturation and/or oxygenation has fallen below a threshold (e.g., if the patient is experiencing hypoxemia). A re-oxygenation routine may include an initial step of rapidly oxygenating the patient, followed by a reduction of oxygenation to make the oxygenation process more gradual. For instance, after the initial step of rapid oxygenation, the patient may be oxygenated with oxygen at an atmospheric level. The system may dynamically adjust the ratio of delivered oxygen versus delivered air, the duration of oxygenation, and the incidence of oxygenation. The system may also adjust the automated delivery of one or more drugs to the patient based on the patient's condition and/or the state of re-oxygenation.

### BACKGROUND

Patient monitoring systems may be used to monitor physiological parameters of patients undergoing diagnostic procedures, surgical procedures, and/or various other types of medical procedures. In some settings, a nurse or technician in a pre-procedure room may prepare a patient for an upcoming procedure. This preparation may include connecting monitors to the patient for the purpose of obtaining baseline data to be used in the procedure. Such monitors may include a blood pressure monitor and pulse oximetry monitor, among others. Blood pressure readings may be taken by a blood pressure cuff, whereby a nurse or technician secures the cuff around a patient's arm and uses a device to pump air into the cuff. Once the reading from the cuff stabilizes, the nurse or technician may have to manually record the data (e.g., handwritten on a sheet of paper or typed into a portable electronic device), and save this information for later reference during the procedure and eventually, for a patient report. For the nurse or technician to take a pulse oximeter reading, he or she may have to boot up the pulse oximeter module, secure a pulse oximeter probe upon the patient, and take a reading of the patient. This reading may also be written down on paper or otherwise be manually recorded for later use. Once it is determined the patient is ready for the procedure, the nurse or technician may have to disengage the blood pressure cuff and pulse oximetry probes from the patient, so the patient can be transported from the pre-procedure room to the procedure room.

After the patient enters the procedure room and before the procedure begins, several tasks may be needed to prepare the patient for the procedure. The nurse or technician may have to reconnect both blood pressure and pulse oximetry readers before the procedure can begin. In addition to blood pressure and pulse oximetry, other connections such as, for example, capnography, supplemental oxygen, and electrocardiogram may be required. A great deal of time may be required to connect the physiological monitors to the patient and to connect the physiological monitors to the monitoring system. In some such instances, the nurse or technician must spend time reconnecting the same kinds of physiological monitors that were previously connected to the patient in the pre-procedure room. The time it takes to make these connections may occupy valuable procedure room time, thus decreasing practice efficiency.

In various settings, it may also be desirable to deliver drugs to a patient during a procedure, such as via an IV and/or face mask, etc. Such drugs may include sedatives, anelgesics, amnestics, etc. In some instances, such drugs may be selected and/or combined to place a patient in a state of “conscious sedation” (in lieu of simply rendering a patient completely unconscious through a general anesthetic). Certain systems may also be used to automate the delivery of such drugs. For instance, such systems may be located in the same room where a medical procedure is performed, and may be coupled with a physiological monitoring system to automatically tailor the delivery of drugs based on patient parameters detected by the monitoring system. Examples of such systems are disclosed in U.S. Pat. No. 6,745,764, entitled “Apparatus and Method for Providing a Conscious Patient Relief from Pain and Anxiety Associated with Medical or Surgical Procedures,” issued Jun. 8, 2004, the disclosure of which is incorporated by reference herein; U.S. Pat. No. 7,833,213, entitled “Patient Monitoring and Drug Delivery System and Method,” issued Nov. 16, 2010, the disclosure of which is incorporated by reference herein; U.S. Pat. No. 7,935,081, entitled “Drug Delivery Cassette and a Medical Effector System,” issued May 3, 2011, the disclosure of which is incorporated by reference herein; U.S. Pat. No. 9,092,559, entitled “Drug Delivery System with Open Architectural Framework,” issued Jul. 28, 2015, the disclosure of which is incorporated by reference herein; U.S. Pub. No. 2009/0292179, entitled “Medical System having a Medical Unit and a Display Monitor,” published Nov. 26, 2009, the disclosure of which is incorporated by reference herein; and U.S. Pub. No. 2010/0010433, entitled “Medical System which Controls Delivery of a Drug,” published Jan. 14, 2010, the disclosure of which is incorporated by reference herein.

An important aspect of patient monitoring during all stages of a procedure may involve tracking a patient's oxygen saturation and providing oxygenation and/or changes in drug delivery in response to unfavorable conditions. In some instances, when oxygen desaturation is detected, conventional wisdom may suggest that oxygen is most critical at the time of desaturation and when the patient takes the first breath from a prolonged apnea episode, such that a clinician may be inclined to provide a high flow of oxygen in response to an oxygen desaturation condition. However, there may be instances where rapid re-oxygenation of organs from a hypoxic condition can result in injuries.

While a variety of systems have been made and used for monitoring patients and delivering drugs and oxygen to patients, it is believed that no one prior to the inventor(s) has made or used the technology as described herein.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a perspective view of an exemplary patient monitoring and drug delivery system;

FIG. 2 depicts a perspective view of the patient monitoring unit of the system of FIG. 1;

FIG. 3 depicts a perspective view of the drug delivery unit of the system of FIG. 1;

FIG. 4 depicts a block diagrammatic view of the system of FIG. 1 with additional exemplary components;

FIG. 5 depicts a flowchart of exemplary steps performed to monitor and adjust oxygenation to address hypoxemia;

FIG. 6 depicts a flowchart of exemplary steps performed to configure a system to manage oxygenation during a hypoxemia event;

FIG. 7 depicts a flowchart of exemplary steps performed to maintain optimal oxygenation during a hypoxemia event; and

FIG. 8 depicts flowchart of exemplary alternative steps performed to maintain optimal oxygenation to address hypoxemia.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview

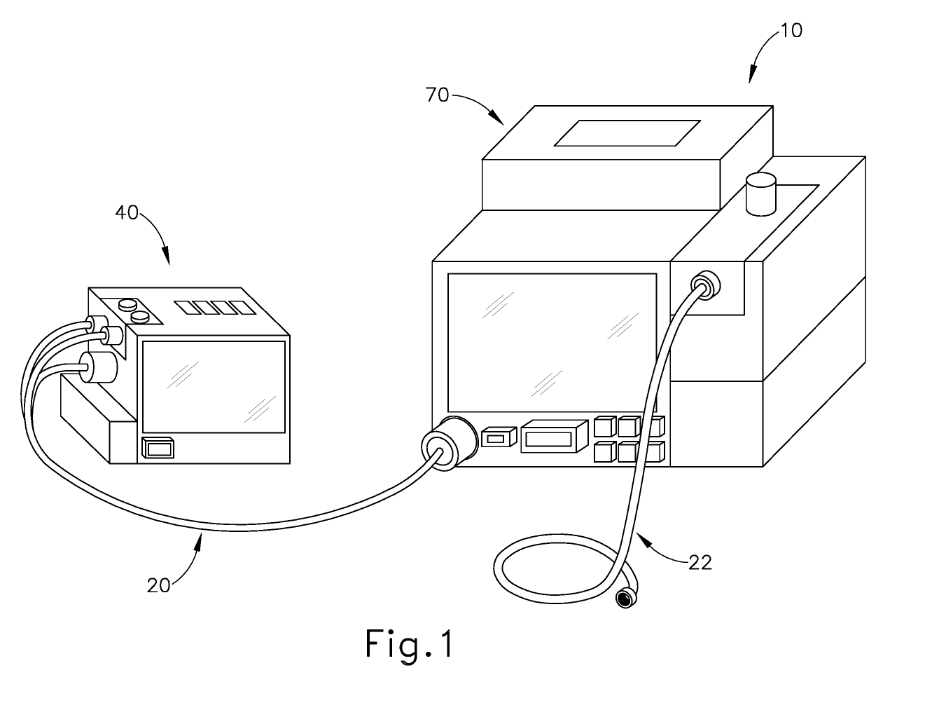

FIG. 1 shows an exemplary patient care system (10) comprising a bedside monitor unit (BMU) (40) and a procedure room unit (PRU) (70). One exemplary use of patient care system (10) is to monitor patient parameters and deliver sedative, analgesic, and/or amnestic drugs to a conscious, non-intubated, spontaneously-ventilating patient undergoing a diagnostic procedure, surgical procedure, or other medical procedure by a physician. This use is not exhaustive of all of the potential uses of the invention but will be used to describe examples herein. BMU (40) and PRU (70) are connected via communication cable (20). Communication cable (20) provides means for transmitting electronic data as well as various hydraulic signals and gases between BMU (40) and PRU (70). For instance, communication cable (20) may include a plurality of pneumatic tubes and a plurality of electrical wires, all integrated within a single sheath or cable. Communication cable (20) may be removed from both BMU (40) and PRU (70) to facilitate practice efficiency and user convenience. BMU (40) and PRU (70) are free to move independently of each other if communication cable (20) is not in place. This allows for mobility of each unit independent of the other; this feature is especially important in hospitals that have a great deal of medical procedures and there is little time to connect patients to monitors. BMU (40) and PRU (70) preferably accommodate an external oxygen source that is intended to provide supplemental oxygen to the patient during the course of a surgical procedure if the clinician so desires. An IV tube set (22) is shown connected to PRU (70) and delivers sedative or amnestic drugs to a patient during a surgical procedure.

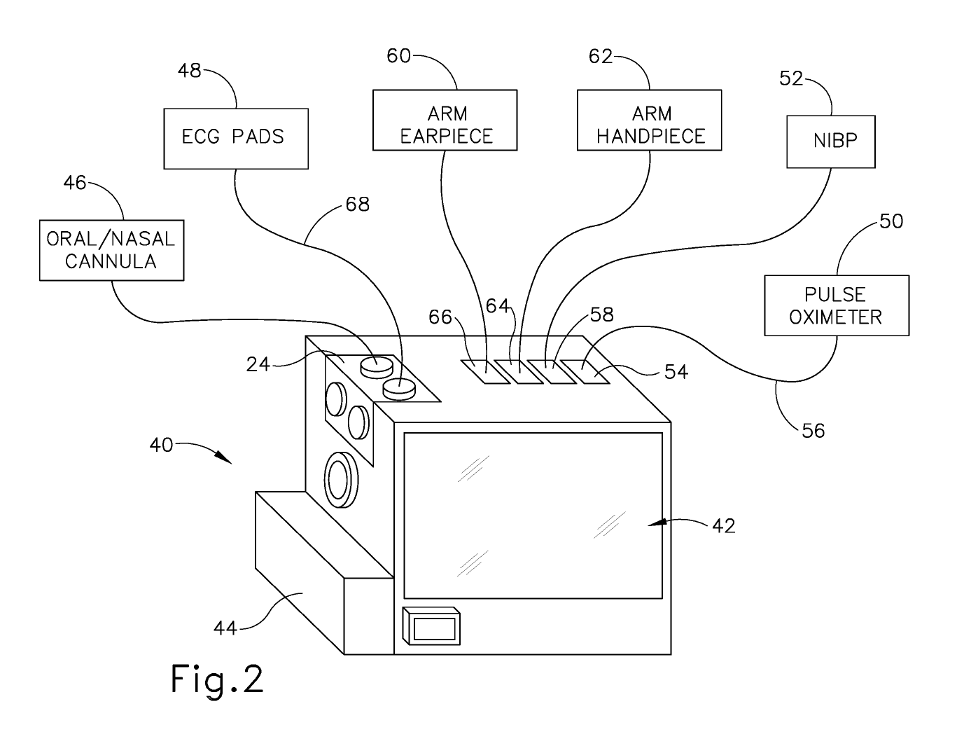

BMU (40) serves as a patient monitoring unit, monitoring various physiological parameters of a patient. As shown in FIG. 2, BMU (40) is compact and portable so it requires relatively little effort to move from one room to another. In some versions, BMU (40) could mount upon either an IV pole or a bedrail; this would free the clinician from the burden of carrying the unit wherever the patient needs to be transported. BMU (40) is small and light enough to be held in the hand of a nurse or technician. BMU (40) allows the user to input information via a touch screen assembly (42) or a simple keypad, etc. Touch screen assembly (42) is provided as an overlay on a display device that is integrated into one surface of BMU (40), and that displays patient and system parameters, and operational status of BMU (40). An exemplary bedside touch screen assembly (42) is a 5.25″ resistive touch screen manufactured by MicroTech mounted upon a 5.25″ color LCD screen manufactured by Samsung. Other suitable forms that a display screen and touch screen may take will be apparent to those of ordinary skill in the art in view of the teachings herein. An attending nurse or physician may enter patient information such as, for example, patient weight and a drug dose profile into BMU (40) by means of bedside touch screen assembly (42). A BMU battery (44) is fixedly attached to the BMU (40) and comprises a standard rechargeable battery such as, for example, Panasonic model no. LC-T122PU, that is capable of supplying sufficient power to run BMU (40) for an extended period of time. In some versions, BMU battery (44) can be recharged while BMU (40) is connected to PRU (70) via communication cable (20) or can be charged directly from an independent power source. Various suitable ways in which battery (44) may be charged will be described in greater detail below in section III.A.; while still other suitable ways will be apparent to those of ordinary skill in the art in view of the teachings herein. Similarly, various suitable forms that battery (44) may take, as well as various suitable compositions thereof, will be apparent to those of ordinary skill in the art in view of the teachings herein.

As shown in FIG. 2, BMU (40) may be connected to a plurality of patient sensors and peripherals used to monitor patient vital signs and deliver supplemental oxygen to the patient. Oral nasal cannula (46) delivers oxygen from an external oxygen source and collects samples of exhaled gas. Oral nasal cannula (46) is removably attached to cable pass-through connection (24). Cable pass-through connection (24) sends the signal obtained by oral nasal cannula (46) directly to a capnometer (e.g., a CardioPulmonary Technologies CO2WFA OEM) in PRU (70) and preferably via communication cable (20) (FIG. 1). The capnometer measures the carbon dioxide levels in a patient's inhalation/exhalation stream via a carbon dioxide-sensor as well as measuring respiration rate. Also attached to the cable pass-through connection (24) is a standard electrocardiogram (ECG) (48), which monitors the electrical activity in a patient's cardiac cycle. The ECG signals are sent to the PRU (70) where the signals are processed. A pulse oximeter probe (50) (e.g., by Dolphin Medical) and a non-invasive blood pressure (NIBP) cuff (52) are also connected to BMU (40) in the present example. Pulse oximeter probe (50) measures a patient's arterial saturation and heart rate via an infrared diffusion sensor. The data retrieved by pulse oximeter probe (50) is relayed to pulse oximeter module (54) (e.g., by Dolphin Medical) by means of pulse oximeter cable (56). The NIBP cuff (52) (e.g., a SunTech Medical Instruments PN 92-0011-00) measures a patient's systolic, diastolic, and mean arterial blood pressure by means of an inflatable cuff and air pump (e.g., by SunTech Medical), also incorporated as needed. NIBP cuff (52) is removably attached to NIBP module (58) located on BMU (40).

In the present example, a patient's level of consciousness is detected by means of an Automated Response Monitor (ARM), though like various other components described herein, an ARM is merely optional and is not required. An exemplary ARM is disclosed in U.S. Pub. No. 2005/0070823, entitled “Response Testing for Conscious Sedation Involving Hand Grip Dynamics,” published Mar. 31, 2005, the disclosure of which is incorporated by reference herein. The ARM of the present example comprises a query initiate device and a query response device. The ARM operates by obtaining the patient's attention with the query initiate device and commanding the patient to activate the query response device. The query initiate device may comprise any type of stimulus device such as a speaker via an earpiece (60), which provides an auditory command to a patient to activate the query response device. The query response device of the present example comprises is a handpiece (62) that can take the form of, for example, a toggle or rocker switch or a depressible button or other moveable member hand held or otherwise accessible to the patient so that the member can be moved or depressed by the patient upon the patient's receiving of the auditory signal or other instruction to respond. Alternatively, a vibrating mechanism may be incorporated into handpiece (62) that cues the patient to activate the query response device. For instance, in some versions, the query initiate device comprises a cylindrical handheld device (62), containing a small 12V DC bi-directional motor enabling the handheld device to vibrate the patient's hand to solicit a response.

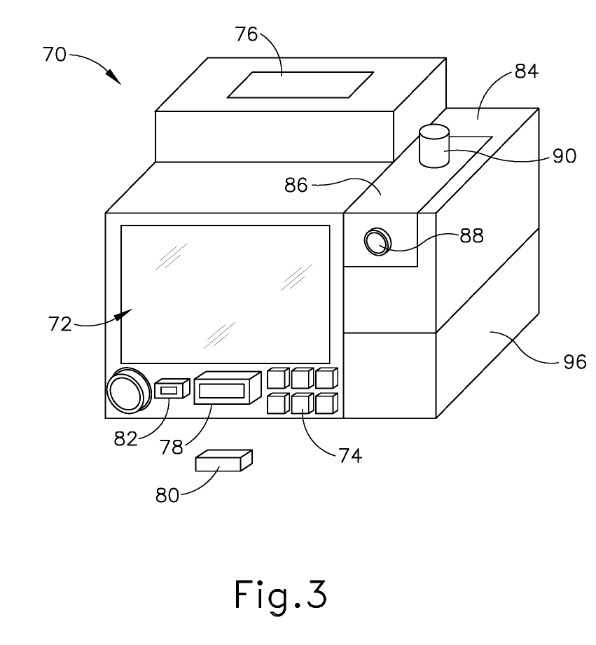

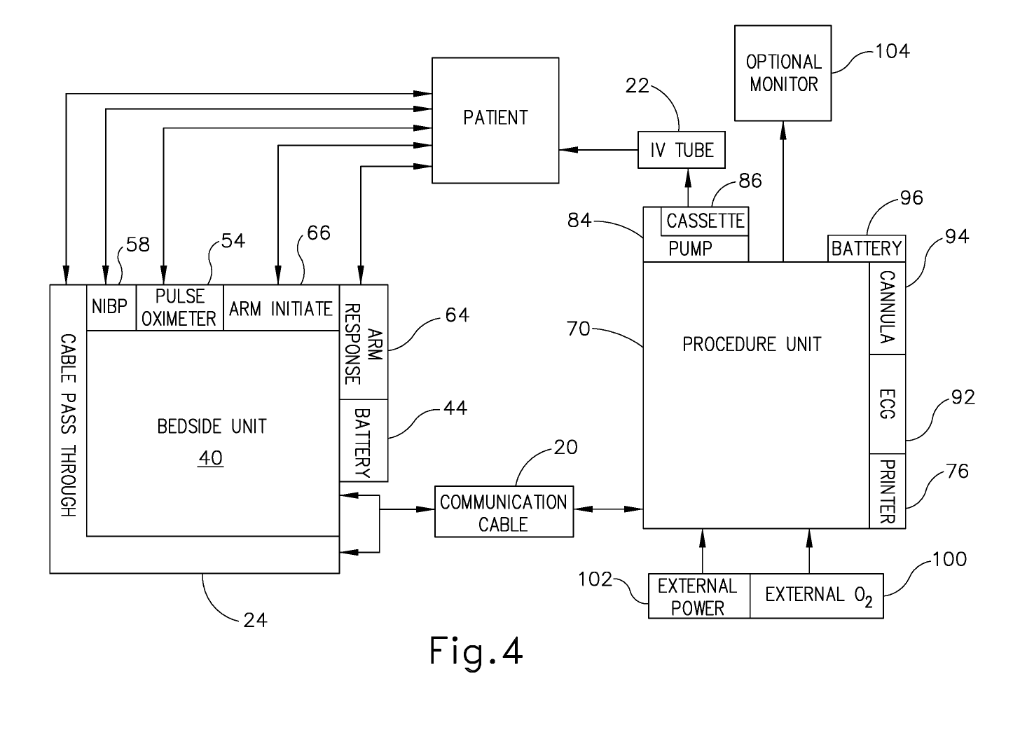

After the query is initiated, the ARM generates signals to reflect the amount of time it took for the patient to activate the query response device in response to the query initiate device. These signals are processed by a logic board located inside BMU (40) and are displayed upon either bedside touch screen assembly (42), procedure touch screen assembly (72) (FIG. 3), and/or an optional monitor 104 (FIG. 4). The amount of time needed for the patient to respond to the query gives the clinician an idea as to the sedation level of the patient. The ARM has two modules in this example, including a query response module (64) and a query initiate module (66), collectively referred to as the ARM modules (64, 66). ARM modules (64, 66) have all the necessary hardware to operate and connect the query response device (62) and the query initiate device (60) to BMU (40).

In some versions monitoring modules (54, 58, 64, 66) are easily replaceable with other monitoring modules in the event of malfunction or technological advancement. These modules (54, 58, 64, 66) include all of the necessary hardware to operate their respective peripherals. The above-mentioned patient modules (54, 58, 64, 66) are connected to a microprocessor-based electronic controller or computer (MLB) located within each of the PRU (70) and BMU (40). The electronic controller or main logic board comprises a combination of available programmable-type microprocessors and other “chips,” memory devices and logic devices on various board(s) such as, for example, those manufactured by Texas Instruments (e.g., XK21E) and National Semiconductor (e.g., HKL72), among others. Various other suitable forms that modules (54, 58, 64, 66) and associated electronics may take will be apparent to those of ordinary skill in the art in view of the teachings herein.

Once BMU (40) and PRU (70) are connected via communication cable (20), ECG and capnography may be monitored, and supplemental oxygen may be delivered to the patient. It should be understood, however, that these connections may be made in the pre-procedure room to increase practice efficiency. By making these connections in the pre-procedure room, less time may be required in the procedure room connecting capnography, ECG and supplemental oxygen to PRU (70). Oral nasal cannula (46) and ECG leads (68) are connected directly to cable pass-through connection (24). Cable pass-through connection (24), located on BMU (40), is essentially an extension of communication cable (20), which allows the signals from ECG leads (68) and oral nasal cannula (46) to bypass BMU (40) and be transferred directly to PRU (70). It will be evident to those skilled in the art, however, that the BMU (40) could be configured to accept the ECG (48) and oral/nasal cannula (46) signals and process the signals accordingly to provide the information on screen (42) and supplemental oxygen to the patient in the pre-procedure room. Other examples of components, features, and functionality that may be incorporated into BMU (40) will be described in greater detail below; while still further examples of components, features, and functionality that may be incorporated into BMU (40) will be apparent to those of ordinary skill in the art in view of the teachings herein.

Referring now to FIG. 3, PRU (70) allows a physician to safely deliver drugs, such as sedative, analgesic, and/or amnestic drugs to a patient, and monitor the patient during a medical procedure. Procedure touch screen assembly (72) comprises a display device that is integrated into the surface of PRU (70), which displays patient and system parameters, and operation status of PRU (70). In some versions, procedure touch screen assembly (72) comprises a 15″ resistive touch screen manufactured by MicroTech mounted upon a 15″ color LCD screen manufactured by Samsung. Other suitable forms that a display screen and touch screen may take will be apparent to those of ordinary skill in the art in view of the teachings herein. It should be noted that, in the present example, procedure touch screen assembly (72) is the primary display and user input means, and is significantly larger than the bedside touch screen assembly (42) and is capable of displaying more detailed information. In addition to procedure touch screen assembly (72), the user may input information into PRU (70) by means of drug delivery controls (74). Drug delivery controls (74), such as buttons, dials, etc., are located on one side of PRU (70) and allow the clinician to change various system parameters and bypass procedure touch screen assembly (72). A printer (76) is integrally attached to the top of PRU (70). Printer (76) allows the clinician to print a patient report that includes patient data for pre-op and the procedure itself. The combination of printing a patient report and the automatic data logging features may decrease the amount of time and effort a nurse or technician must spend regarding patient condition during the course of a procedure. Printer (76) receives data signals from a printer interface (e.g., Parallel Systems CK205HS), which is located on the main logic board. Printer (76) may comprise a thermal printer (e.g., Advanced Printing Systems (APS) ELM 205HS) and/or any other suitable type of printer. It should also be understood that printer (76) may be remote from PRU (70) and may even be omitted altogether, if desired.

Memory card reader (78), which includes a slot in the outer casing of PRU (70), allows flash memory card (80) to be inserted and removed from PRU (70). Flash memory card (80) is a solid-state storage device used for easy and fast information storage of the data log generated by PRU (70). The data is stored so that it may be retrieved from flash memory card (80) at a later time. In some versions, memory card reader (78) accepts flash memory card (80) containing software to upgrade the functionality of patient care system (10). Again, as with other components described herein, memory card reader (78) may be modified, substituted, supplemented, or omitted as desired. In the present example, memory card reader (78) is supplemented with a data port (82). Data port (82) may include, but is not limited to, a standard serial port, a USB port, a RS232 port, an Ethernet port, or a wireless adapter (e.g., using IEEE 802.11n/g/b/a standard, etc.). Data port (82) may be used to link PRU (70) to an external printer to print a patient report or to transfer electronic files to a personal computer or mainframe. A merely illustrative example of how data port (82) may be used to communicate with a centralized network system component will be described in greater detail below in section III. B., while still other suitable examples will be apparent to those of ordinary skill in the art in view of the teachings herein.

PRU (70) delivers fluid to a patient via an infusion pump, such as a peristaltic infusion pump (84) (e.g., by B-Braun McGaw). Peristaltic infusion pump (84) is integrally attached to PRU (70), and uses peristaltic fingers to create a wavelike motion to induce fluid flow inside a flexible tube connected to a fluid reservoir. A drug cassette (86) is a generally rectangular shaped structure that is placed adjacent to peristaltic infusion pump (84). Drug cassette (86) of this example is made of a rigid thermoplastic such as, for example, polycarbonate. Drug cassette (86) has an internal cavity that houses IV tubing (22) made of a flexible thermoplastic such as, for example, polypropylene (e.g., Kelcourt). Drug cassette (86) receives tubing (22) via a port (88) and accurately and reliably positions exposed IV tubing (22) in contact with the peristaltic fingers of peristaltic infusion pump (84). IV tube set (22) attaches to a fluid vial (90), and a portion of the length of IV tube set (22) is contained within drug cassette (86). Another portion of IV tube set (22) lies external to drug cassette (86) to facilitate the interaction with peristaltic pump (84). IV tubing (22) is coiled within drug cassette (86) and has a length to reach a patient removed from the PRU (70). A fluid detection sensor (not shown) may be mounted to an inner wall of drug cassette (86). Such a fluid detection sensor may comprise any one of known fluid sensors, such as the MTI-2000 Fotonic Sensor, or the Microtrak-II CCD Laser Triangulation Sensor both by MTI Instruments Inc. IV tube set (22) may run through the fluid detection sensor before exiting drug cassette (86). PRU (70) may include features operable to prime IV tubing (22) with relative ease for a user. Various examples of how such priming may be provided are disclosed in U.S. Pat. No. 7,833,213, the disclosure of which is incorporated by reference herein.

In the present example, drug cassette (86) includes just one vial (90). However, it should be understood that some versions of drug cassette (86) may include several vials (90). Such vials (90) may include the same drug. Alternatively, a plurality of vials (90) associated with a single drug cassette (86) may include a variety of different kinds of drugs. In other words, a single drug cassette (86) may be used to selectively deliver two or more drugs simultaneously and/or in a particular sequence. While vials (90) are used in the present example, it should be understood that any other suitable type of container may be used as will be understood by those of ordinary skill in the art in view of the teachings herein. It should also be understood that some versions of PRU (70) may be configured to receive two or more drug cassettes (86). Each such drug cassette (86) may be associated with a single drug (e.g., different drug cassettes (86) used for different drugs), or each drug cassette (86) may be associated with a combination of drugs (e.g., different drug cassettes (86) used for different combinations of drugs).

FIG. 4 shows how components of system (10) interface with each other and with a patient. While not shown in FIG. 3, FIG. 4 shows how PRU (70) includes an integral ECG module (92) and integral cannula module (94). ECG module (92) is coupled with ECG (48) via ECG leads (68) extending from pass-through connection (24). Cannula module (94) is coupled with oral/nasal cannula (46), also through pass-through connection (24). Like modules (54, 58, 64, 66) described above, modules (92, 94) may be easily replaceable with other monitoring modules in the event of malfunction or technological advancement. Modules (92, 94) may also include all of the necessary hardware to operate their respective peripherals, and may be further coupled with a microprocessor-based electronic controller or computer located within PRU (70) and/or BMU (40).

As also shown in FIG. 4, PRU (70) of the present example is coupled with an external oxygen source (100), an external power source (102), and an external monitor (104). External oxygen source (100) may by regulated by one or more components of PRU (70), which may deliver oxygen from oxygen source (100) to the patient based on one or more parameters sensed by BMU (40), based on drug delivery from cassette (86), and/or based on other factors. External power source (102) may be used as a primary source of power for PRU (70), with a battery (96) being used as a backup power source. Alternatively, battery (96) may be used as a primary source of power for PRU, with external power source (102) being used for backup power and/or to charge battery (96). External monitor (104) may be used to supplement or to substitute the display features of touch screen assembly (42) and/or touch screen assembly (72). For instance, external monitor (104) may display information including patient physiological parameters, status of operation of system (10), warning alerts, etc. PRU (70) and/or BMU (40) may communicate with external monitor (104) via cable, wirelessly (e.g., via RF transmission, etc.), or otherwise. Other examples of components, features, and functionality that may be incorporated into PRU (70) will be described in greater detail below; while still further examples of components, features, and functionality that may be incorporated into PRU (70) will be apparent to those of ordinary skill in the art in view of the teachings herein.

II. Oxygenation Management to Address Hypoxemia

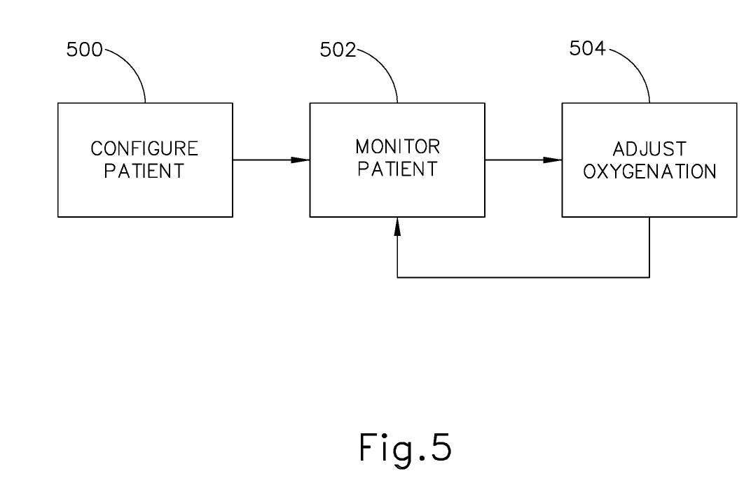

Some conventional systems and practices may utilize high volume oxygen delivery in response to a desaturation event. It may instead be desirable to provide a more conservative and reactive real-time management of re-oxygenation to reduce the occurrence of injury and organ damage relating to the desaturation event. Such an effect can be achieved by, for example, configuring one or both of the BMU (40) or PRU (70) to adjust delivery of oxygen via the oral/nasal cannula (46) in response to receiving data from a monitor, such as NIBP monitor (58), pulse oximeter (54), integral cannula module (94), or another monitor (104). FIG. 5 shows a high level flowchart of exemplary steps that may be performed to monitor and adjust re-oxygenation during a hypoxemia event. Initially, the system may be configured (block 500) for a particular patient. This could include attaching one or more monitors or delivery devices to the patient, including but not limited to the devices of patient care system (10) described above, identifying the patient and their characteristics, receiving manual or automated configurations, or similar processes. Patient monitoring (block 502) may then begin. As monitored data is received, oxygenation may be adjusted (block 504) based upon each piece of data, at a set time interval, or per a group of pieces of data, with the steps of monitoring (block 502) and adjusting (block 504) repeating until a procedure is completed or a hypoxemia event is resolved. For example, in a system with high processing speed, each piece of data may be individually processed as it arrives. In less powerful systems, data may be collected over a short period of time, or a set number of pieces of data may be collected, with each collection being aggregated or averaged and processed by the system to more efficiently use available processing cycles.

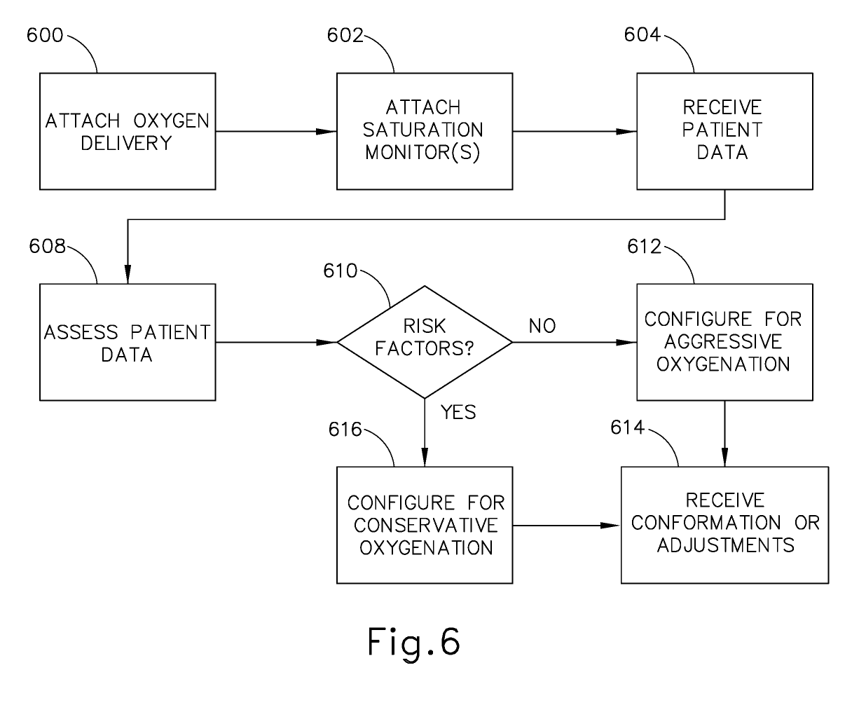

FIG. 6 shows a flowchart of an exemplary set of steps performed to configure a system (500), such as one or both of the BMU (40) and PRU (70), to manage oxygenation of a patient during a hypoxemia event. An oxygen delivery device, such as an oral/nasal cannula (46) may be attached to an oxygen source and to a patient (block 600) so that the patient may receive oxygen in higher concentration or volume than typical atmospheric levels. Oxygenation monitors and/or oxygen saturation monitors may also be attached to the patient (block 602), and may include such monitors as the NIBP monitor (58), cannula module (94), pulse oximeter (54), and/or other device(s) (104) (e.g., an invasive monitor that is configured to assess the patient's arterial blood oxygen level (PaO₂)). Such monitors may be stand alone devices in some versions; or may be built in or attached to another device such as the BMU (40) or PRU (70). Patient data may be received (block 604) via a manual input by a clinician or assistant, or by an automatic process whereby the system communicates with a medical records server and database via a wireless or wired communication. Once patient data is received (block 604), the patient data is assessed (block 608) by the system.

Assessment of patient data (block 608) may include an automated evaluation of the risks and physiological factors for a particular patient that impact hypoxemia and re-oxygenation. Factors such as age, height, weight, race, and gender may be considered, as well as medical conditions such as sickness or disease affecting the respiratory system (e.g., sleep apnea or COPD), circulatory system, or general health of the patient, past medical events such as prior hypoxemia events, recent surgeries or treatments that might impact hypoxemia, location or altitude of the procedure site, or other similar factors that will be apparent to one of ordinary skill in the art in light of this disclosure. If the patient data, once assessed (block 608), exhibits risk factors (block 610) based upon individual characteristics or combinations of characteristics, the system may be automatically configured for a conservative oxygenation (block 616) plan. For example, advanced age may be a risk factor that results in a conservative configuration by itself, while in other cases, the combination of an unhealthy patient weight and a pre-existing respiratory condition may result in a conservative configuration, and cause oxygen delivery to be adjusted in smaller steps as compared to an aggressive treatment, or may cause oxygen delivery concentration to start at a lower initial level as compared to an aggressive plan. The particular configuration of risk factors may vary greatly on a case by case basis, and risk factors may vary by geographic location, patient, equipment manufacturer, equipment owner, or otherwise.

Where the patient data does not exhibit any risk factors (block 610), the system may be configured for an aggressive oxygenation plan (block 612). For example, if a patient appears to be in good health with no negative medical history, an aggressive oxygenation plan may be appropriate, and may start with a higher initial oxygen delivery concentration and may also adjust oxygen delivery in larger steps as compared to a conservative routine. Within aggressive and conservative oxygenation routines there may be more than one routine to address overall high or low levels of risk, specific risk characteristics, or specific configurations of equipment available for a procedure. For example, an aggressive oxygenation routine might include use of a bag mask and a delivery rate of 12 liters per minute or more. A conservative oxygenation routine might include the use of an oral/nasal cannula to deliver oxygen at a rate of around 2-4 liters per minute.

Whether conservative or aggressive, the oxygenation routine may specify such characteristics as an initial oxygen delivery rate, concentration, or volume, one or more intermediate or final oxygen delivery concentrations, alarm limits or thresholds on various physiological factors that may result in an alarm sounding when sensed by the monitor devices, drug delivery rates for one or more attached drug cassettes (86), and other similar configurable factors. Once the initial configuration is set, whether conservative or aggressive, it may be displayed upon a viewable screen of the BMU (40), PRU (70), or other display. This preview may be viewed and then the display device (42, 72) or another input device may receive an input from a clinician revising or confirming the initial configuration (block 614). For example, if an aggressive configuration is chosen for a particular patient by the system, and a clinician feels that the initial oxygen delivery concentration is too high, or that the intermediate oxygen delivery steps are not varied enough, one or more of those characteristics may be manually configured and adjusted, and the modified configuration may be confirmed (block 614).

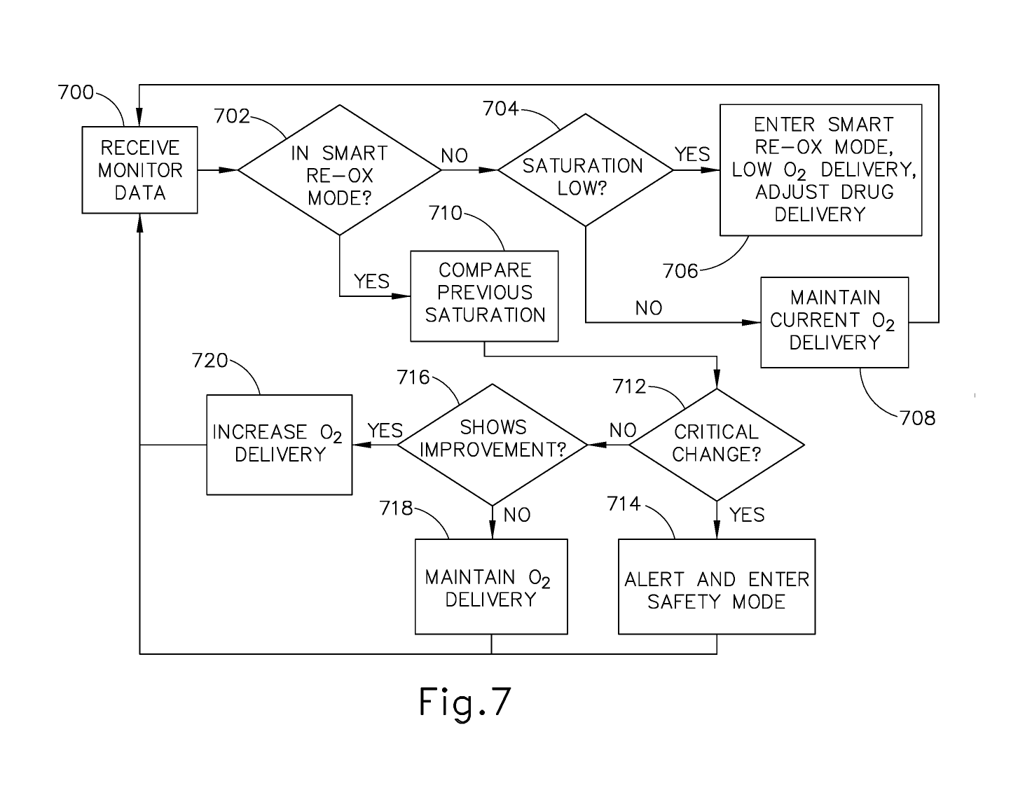

FIG. 7 shows a set of exemplary steps that may be performed to maintain optimal oxygenation during a hypoxemia event by adjusting oxygen delivery (504) in response to monitored data (502). In this example, these steps are shown as a recurring set of steps that may be looped through for the duration of a procedure or event. The set of steps may be performed each time data is received from a monitoring device, or may be performed on a batch of several pieces of data gathered over a defined time interval or defined batch size, or may be performed as quickly as the associated hardware is able to receive and process such data. Additionally, each iteration of these steps may be performed one at a time in a serial manner; or multiple iterations of these steps may be performed in parallel, depending upon the hardware capabilities of a specific implementation. For each iteration of steps, monitor data is received (block 700) from the monitor devices and sensors. Initially, the system will be in a static oxygen delivery mode as opposed to a smart re-oxygenation mode (block 702). When the system is operating in static oxygenation mode, it may determine, at each iteration, whether oxygen saturation is below a predetermined threshold (block 704). This could include a determination of oxygen saturation via a non-invasive sensor such as a pulse oximeter, a determination of actual arterial blood oxygen level (PaO₂) via an invasive measurement, or both. If the oxygen saturation is above the threshold, current oxygen delivery will be maintained (block 708) until the next set of monitor data is received (block 700).

If the oxygen saturation falls below the threshold, indicating that oxygen saturation is low (block 704), the system may enter a dynamic re-oxygenation mode, that may be referred to as smart re-oxygenation mode (block 706). In this smart re-oxygenation mode (block 706), the system may reduce the delivery of oxygen to atmospheric levels. In some versions, the system may also adjust drug delivery in the smart re-oxygenation mode. For instance, in the smart re-oxygenation mode (block 706), the system may decrease the flow of drugs that negatively impact a hypoxemia event and/or increase the flow of drugs that positively impact a hypoxemia event. After entering the smart re-oxygenation mode (block 706), the system will receive additional monitor data (block 700) and a new iteration may begin.

Once the system is operating in smart re-oxygenation mode (block 702), each set of monitor data received will be compared to a previous set of monitor data (block 710) to determine the difference between the currently measured oxygen saturation level and the previously measured oxygen saturation level. If the compared differenced (block 710) indicates a critical change in oxygenation (block 712), the system may generate alerts to a physician or other staff and may enter a safety mode (block 714) that causes an adjustment in oxygen delivery or drug delivery in an attempt to prevent injury or degradation of the patient's condition until a clinician can respond to the alarm. For example, if, while in smart re-oxygenation mode, a current set of monitor data shows that saturation levels have dropped or increased drastically, unexpectedly, or unpredictably from a previous value (block 712), rather than continuing the smart re-oxygenation routine the system may generate visual and audible alerts so that a clinician can verify that the monitors are still attached and functioning properly; or provide immediate diagnosis of a change in the patient's status that caused the unexpected drop in saturation.

If no critical change is detected (block 712), the current monitor data is compared to the previous monitor data to determine if the patient's oxygen saturation shows improvement (block 716). If oxygen saturation does not show improvement, the current level of oxygen may be maintained (block 718). If the patient's oxygen saturation does show improvement (block 716), oxygen delivery maybe increased from the current oxygen delivery concentration to a next intermediate oxygen delivery concentration, or to a final oxygen delivery concentration (block 720). After oxygen delivery is increased (block 720) or maintained (block 718), the system proceeds to a further iteration of steps. Operating in this manner, the system may begin smart re-oxygenation mode delivering oxygen at an initial delivery concentration of low or atmospheric oxygen levels (block 706). A subsequent monitor data set may indicate that the patient's saturation level has not improved (block 716) and cause the system to maintain the current atmospheric oxygen delivery concentration (block 718), as opposed to delivering oxygen at high concentration despite the lack of improvement and potentially causing injury. A further subsequent monitor data may indicate that the patient's saturation level has improved (block 720), and oxygen delivery may be increased by a step now that the patient's vital signs are responding positively to re-oxygenation efforts. In this manner, the system may start at an atmospheric level and then gradually increase oxygen delivery throughout a number of intermediate oxygen delivery steps, allowing it to automatically maintain the highest level of oxygen delivery that results in an improved condition for the patient.

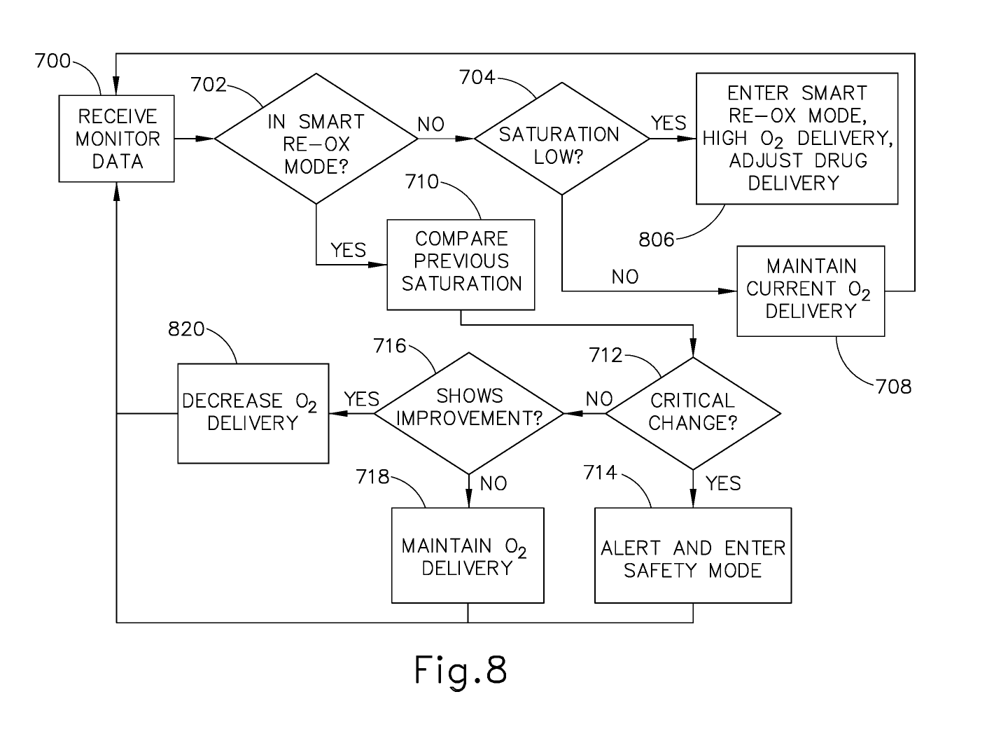

FIG. 8 shows another exemplary set of steps that may be performed to maintain a smart re-oxygenation mode. The method depicted in FIG. 8 is substantially similar to the method depicted in FIG. 7. In the example shown in FIG. 8, when oxygen saturation levels are low (block 704) the system may enter smart re-oxygenation mode, causing it to begin delivering oxygen at a high delivery concentration, while also adjusting drug delivery rate appropriately (block 806). When a subsequent monitor data shows improvement in oxygen saturation (block 716), oxygen delivery may be maintained at current levels (block 718), since the current level of re-oxygenation is resulting in an improved condition of the patient. When a subsequent monitor data shows no improvement in oxygen saturation (block 716), the system may decrease the current oxygen delivery concentration by an intermediate step (block 820). Operating in this manner, the system may begin at a high oxygen delivery concentration at the start of a hypoxemia event, and then may decrease oxygen delivery by a number of intermediate steps until it reaches an oxygen delivery concentration that results in improvement in the patient's oxygen saturation.

In some versions, the system may, in situations where a patient is not responding to a current smart re-oxygenation routine, switch from one re-oxygenation mode to another. For example, if a patient does not respond to the re-oxygenation routine of FIG. 7 after a certain number of iterations, the system may automatically, or through manual interventions, switch to the re-oxygenation routine of FIG. 8. In some versions, automated changes, such as a change in re-oxygenation routines or even an automated increase or decrease in oxygen delivery may require a confirmation from a clinician. In some versions, monitor data may also include or be used to create an oxygen-hemoglobin disassociation curve, which may be used by the system instead of or in addition to oxygen saturation data when determining a course of action. Specifically, prior to a point of inflection on the curve, the oxygen may be dramatically increased to prevent the patient from transitioning to a steeper part of the curve. Additionally, once a patient has a very low blood gas present, the system may use a more conservative re-oxygenation approach to reduce the possibility of the introduction of reactive oxygen species into the blood stream.

It should be understood from the foregoing that a patient monitoring system may be configured to monitor oxygen saturation and/or oxygenation of a patient's blood. In particular, the system may be configured to re-oxygenate the patient in response to a determination that the patient's oxygen saturation and/or oxygenation has fallen below a threshold (e.g., if the patient is experiencing hypoxemia). A re-oxygenation routine may include an initial step of rapidly oxygenating the patient, followed by a reduction of oxygenation to make the oxygenation process more gradual. For instance, after the initial step of rapid oxygenation at 12 liters per minute, the patient may be oxygenated with oxygen at a lower level of 2 liters per minute, or atmospheric levels. The system may dynamically adjust the ratio of delivered oxygen versus delivered air, the duration of oxygenation, and the incidence of oxygenation. The system may also adjust the automated delivery of one or more drugs to the patient based on the patient's condition and/or the state of re-oxygenation. Another example may be a patient that is presented from a code with low oxygenation, and after an initial assessment, the system may attempt an initial rapid process (12 liters per minute) to improve the initial assessment of the patient, and then slow down the oxygenation process at 2 liters per minute until nominal levels are reached.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a monitoring device, wherein the monitoring device is operable to monitor one or both of oxygenation of a patient or oxygen saturation of a patient; and (b) an oxygen delivery device comprising: (i) an oxygen delivery output; and (ii) an oxygen delivery controller; wherein the oxygen delivery controller is configured to receive a first set of saturation data from the monitoring device; wherein the oxygen delivery controller is configured to enter a dynamic re-oxygenation mode in response to receiving a set of saturation data indicating that a patient is experiencing hypoxemia; wherein the oxygen delivery controller is configured to, upon entering the dynamic re-oxygenation mode, deliver oxygen at an initial delivery concentration via the oxygen delivery output; wherein the oxygen delivery controller is further configured to receive a second set of saturation data from the monitoring device; and wherein the oxygen delivery controller is further configured to perform a transition from the initial delivery concentration to a first transitional delivery concentration of a set of transitional delivery concentrations based upon a change comparison of the second set of saturation data and the first set of saturation data.

### Example 2

The apparatus of Example 1, further comprising a patient server, wherein the patient server is configured to store a set of patient data for the patient, wherein the oxygen delivery controller is configured to receive the set of patient data, and wherein the oxygen delivery controller is configured to determine, based upon the set of patient data: (A) the initial delivery concentration, (B) the set of transitional delivery concentrations, and (C) a change threshold; wherein the oxygen delivery controller is configured to exit the dynamic re-oxygenation mode and enter a safety mode when the difference between a current saturation level and a previous saturation level exceeds the change threshold.

### Example 3

The apparatus of Example 2, further comprising an alarm device, the alarm device comprising one or more of an audible alarm and a visual alarm, wherein the alarm device is configured to activate in response to the oxygen delivery controller entering the safety mode.

### Example 4

The apparatus of any one or more of Examples 1 through 3, wherein the oxygen delivery controller is configured to perform the transition from the initial delivery concentration to a first transitional delivery concentration in response to the change comparison indicating that the patient's oxygen saturation level has improved.

### Example 5

The apparatus of Example 4, wherein the initial delivery concentration is about 21% oxygen, and wherein each of the set of transitional delivery concentrations is greater than about 21% oxygen.

### Example 6

The apparatus of any one or more of Examples 1 through 5, wherein the oxygen delivery controller is configured to perform the transition from the initial delivery concentration to a first transitional delivery concentration in response to the change comparison indicating that the patient's oxygen saturation level has not improved.

### Example 7

The apparatus of Example 6, wherein the initial delivery concentration is above about 21% oxygen, and wherein each of the set of transitional delivery concentrations is greater than about 21% oxygen and less than the initial delivery concentration.

### Example 8

The apparatus of any one or more of Examples 1 through 7, wherein the monitoring device comprises a pulse oximeter, wherein the oxygen delivery output comprises an oral nasal cannula, and wherein the oxygen delivery controller comprises one or both of a bedside monitoring unit or a procedure room unit.

### Example 9

The apparatus of any one or more of Examples 1 through 8, wherein the first set of saturation data, the second set of saturation data, and each subsequent set of saturation data comprise a set of oxygen disassociation data, wherein the oxygen delivery controller is configured to create an oxygen disassociation curve from the oxygen disassociation data.

### Example 10

The apparatus of any one or more of Examples 1 through 9, further comprising a drug delivery device, wherein the drug delivery device is configured to adjust a rate at which a drug is delivered to the patient based upon the dynamic re-oxygenation mode and the change comparison.

### Example 11

A method comprising: (a) configuring an oxygen delivery device for a patient, the oxygen delivery device comprising an oxygen delivery output and an oxygen delivery controller; (b) configuring a monitoring device, wherein the monitoring device is operable to monitor one or both of oxygenation of the patient or oxygen saturation of the patient; (c) receiving, at the oxygen delivery controller, a first set of saturation data from the monitoring device, the first set of saturation data indicating that the patient is experiencing hypoxemia; (d) in response to receiving the first set of saturation data, causing the oxygen delivery controller to enter a dynamic re-oxygenation mode, wherein entering the dynamic re-oxygenation mode causes the oxygen delivery controller to deliver oxygen at an initial delivery concentration via the oxygen delivery output; (e) receiving a second set of saturation data from the monitoring device; and (f) based upon a change comparison of the first set of saturation data and the second set of saturation data, cause the oxygen delivery controller to perform a transition from the initial delivery concentration to a first transitional delivery concentration of a set of transitional delivery concentrations.

### Example 12

The method of Example 11, wherein configuring an oxygen delivery device for a patient comprises: (i) receiving, from a patient server, a set of patient data for the patient, (ii) identifying one or more re-oxygenation risk factors exhibited by the set of patient data, (iii) configuring the oxygen delivery controller to address the one or more re-oxygenation risk factors, and (iv) receiving a confirmation from a clinician indicating that the oxygen delivery controller configuration is acceptable.

### Example 13

The method of Example 12, wherein configuring the oxygen delivery controller to address the one or more re-oxygenation risk factors comprises: (A) determining the initial delivery concentration, (B) determining the set of transitional delivery concentrations, (C) determining a change threshold, and (D) configuring the oxygen delivery controller to exit the dynamic re-oxygenation mode and enter a safety mode when the difference between a current saturation level and a previous saturation level exceeds the change threshold.

### Example 14

The method of Example 13, further comprising the step causing an alarm device to activate when the oxygen delivery controller enters the safety mode.

### Example 15

The method of any one or more of Examples 11 through 14, wherein the transition occurs when the change comparison indicates that the patient's oxygen saturation level has improved.

### Example 16

The method of Example 15, wherein the initial delivery concentration is about 21% oxygen, and wherein each of the set of transitional delivery concentrations is greater than about 21% oxygen.

### Example 17

The method of any one or more of Examples 11 through 16, wherein the transition occurs when the change comparison indicates that the patient's oxygen saturation level has not improved.

### Example 18

The method of Example 17, wherein the initial delivery concentration is above about 21% oxygen, and wherein each of the set of transitional delivery concentrations is greater than about 21% oxygen and less than the initial delivery concentration.

### Example 19

The method of any one or more of Examples 11 through 18, further comprising the step adjusting a drug delivery rate of a drug being delivered to the patient via a drug delivery device based upon the dynamic re-oxygenation mode and the change comparison.

### Example 20

An apparatus comprising: (a) a monitoring device comprising a pulse oximeter; (b) an oxygen delivery device comprising: (i) an oxygen delivery output, comprising an oral nasal cannula, and (ii) an oxygen delivery controller; (c) a drug delivery device; (d) a patient server; and (e) an alarm device; wherein the oxygen delivery controller is configured to receive a set of patient data from the patient server; wherein the oxygen delivery controller is configured to determine, based upon the patient data: (i) an initial delivery concentration, (ii) a set of transitional delivery concentrations, and (iii) a change threshold, wherein the oxygen delivery controller is configured to receive a first set of saturation data from the monitoring device, the first set of saturation data indicating that a patient is experiencing hypoxemia; wherein the oxygen delivery controller is configured to enter a dynamic re-oxygenation mode in response to receiving the set of saturation data, wherein, upon entering dynamic re-oxygenation mode: (i) the oxygen delivery controller is configured to deliver oxygen at the initial delivery concentration via the oxygen delivery output, wherein the initial delivery concentration is about 21% oxygen, and (ii) the drug deliver unit is configured to adjust the rate at which a drug is delivered to the patient; wherein the oxygen delivery controller is further configured to receive a second set of saturation data from the monitoring device; wherein the oxygen delivery controller is further configured to perform a transition from the initial delivery concentration to a first transitional delivery concentration of the set of transitional delivery concentrations in response to a change comparison of the second set of saturation data and the first set of saturation data indicating that the patient's oxygen saturation level has improved, wherein each of the set of transitional delivery concentrations is greater than about 21% oxygen; and wherein the oxygen delivery controller is configured to exit the dynamic re-oxygenation mode, enter a safety mode, and cause the alarm device to activate when the difference between a current saturation level and a previous saturation level exceeds the change threshold.

IV. Miscellaneous

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a monitoring device, wherein the monitoring device is operable to monitor one or both of oxygenation of a patient or oxygen saturation of a patient; and
(b) an oxygen delivery device comprising:
(i) an oxygen delivery output; and
(ii) an oxygen delivery controller;
wherein the oxygen delivery controller is configured to receive a first set of saturation data from the monitoring device;
wherein the oxygen delivery controller is configured to enter a dynamic re-oxygenation mode in response to receiving a set of saturation data indicating that a patient is experiencing hypoxemia;
wherein the oxygen delivery controller is configured to, upon entering the dynamic re-oxygenation mode, deliver oxygen at an initial delivery concentration via the oxygen delivery output;
wherein the oxygen delivery controller is further configured to receive a second set of saturation data from the monitoring device; and
wherein the oxygen delivery controller is further configured to perform a transition from the initial delivery concentration to a first transitional delivery concentration of a set of transitional delivery concentrations based upon a change comparison of the second set of saturation data and the first set of saturation data.

2. The apparatus of claim 1, further comprising a patient server, wherein the patient server is configured to store a set of patient data for the patient, wherein the oxygen delivery controller is configured to receive the set of patient data, and wherein the oxygen delivery controller is configured to determine, based upon the set of patient data:
(A) the initial delivery concentration,
(B) the set of transitional delivery concentrations, and
(C) a change threshold;
wherein the oxygen delivery controller is configured to exit the dynamic re-oxygenation mode and enter a safety mode when the difference between a current saturation level and a previous saturation level exceeds the change threshold.

3. The apparatus of claim 2, further comprising an alarm device, the alarm device comprising one or more of an audible alarm and a visual alarm, wherein the alarm device is configured to activate in response to the oxygen delivery controller entering the safety mode.

4. The apparatus of claim 1, wherein the oxygen delivery controller is configured to perform the transition from the initial delivery concentration to a first transitional delivery concentration in response to the change comparison indicating that the patient's oxygen saturation level has improved.

5. The apparatus of claim 4, wherein the initial delivery concentration is about 21% oxygen, and wherein each of the set of transitional delivery concentrations is greater than about 21% oxygen.

6. The apparatus of claim 1 wherein the oxygen delivery controller is configured to perform the transition from the initial delivery concentration to a first transitional delivery concentration in response to the change comparison indicating that the patient's oxygen saturation level has not improved.

7. The apparatus of claim 6, wherein the initial delivery concentration is above about 21% oxygen, and wherein each of the set of transitional delivery concentrations is greater than about 21% oxygen and less than the initial delivery concentration.

8. The apparatus of claim 1, wherein the monitoring device comprises a pulse oximeter, wherein the oxygen delivery output comprises an oral nasal cannula, and wherein the oxygen delivery controller comprises one or both of a bedside monitoring unit or a procedure room unit.

9. The apparatus of claim 1, wherein the first set of saturation data, the second set of saturation data, and each subsequent set of saturation data comprise a set of oxygen disassociation data, wherein the oxygen delivery controller is configured to create an oxygen disassociation curve from the oxygen disassociation data.

10. The apparatus of claim 1, further comprising a drug delivery device, wherein the drug delivery device is configured to adjust a rate at which a drug is delivered to the patient based upon the dynamic re-oxygenation mode and the change comparison.

11. A method comprising:
(a) configuring an oxygen delivery device for a patient, the oxygen delivery device comprising an oxygen delivery output and an oxygen delivery controller;
(b) configuring a monitoring device, wherein the monitoring device is operable to monitor one or both of oxygenation of the patient or oxygen saturation of the patient;
(c) receiving, at the oxygen delivery controller, a first set of saturation data from the monitoring device, the first set of saturation data indicating that the patient is experiencing hypoxemia;
(d) in response to receiving the first set of saturation data, causing the oxygen delivery controller to enter a dynamic re-oxygenation mode, wherein entering the dynamic re-oxygenation mode causes the oxygen delivery controller to deliver oxygen at an initial delivery concentration via the oxygen delivery output;
(e) receiving a second set of saturation data from the monitoring device; and
(f) based upon a change comparison of the first set of saturation data and the second set of saturation data, cause the oxygen delivery controller to perform a transition from the initial delivery concentration to a first transitional delivery concentration of a set of transitional delivery concentrations.

12. The method of claim 11, wherein configuring an oxygen delivery device for a patient comprises:
(i) receiving, from a patient server, a set of patient data for the patient,
(ii) identifying one or more re-oxygenation risk factors exhibited by the set of patient data,
(iii) configuring the oxygen delivery controller to address the one or more re-oxygenation risk factors, and
(iv) receiving a confirmation from a clinician indicating that the oxygen delivery controller configuration is acceptable.

13. The method of claim 12, wherein configuring the oxygen delivery controller to address the one or more re-oxygenation risk factors comprises:
(A) determining the initial delivery concentration,
(B) determining the set of transitional delivery concentrations,
(C) determining a change threshold, and
(D) configuring the oxygen delivery controller to exit the dynamic re-oxygenation mode and enter a safety mode when the difference between a current saturation level and a previous saturation level exceeds the change threshold.

14. The method of claim 13, further comprising the step causing an alarm device to activate when the oxygen delivery controller enters the safety mode.

15. The method of claim 11, wherein the transition occurs when the change comparison indicates that the patient's oxygen saturation level has improved.

16. The method of claim 15, wherein the initial delivery concentration is about 21% oxygen, and wherein each of the set of transitional delivery concentrations is greater than about 21% oxygen.

17. The method of claim 11, wherein the transition occurs when the change comparison indicates that the patient's oxygen saturation level has not improved.

18. The method of claim 17, wherein the initial delivery concentration is above about 21% oxygen, and wherein each of the set of transitional delivery concentrations is greater than about 21% oxygen and less than the initial delivery concentration.

19. The method of claim 11, further comprising the step adjusting a drug delivery rate of a drug being delivered to the patient via a drug delivery device based upon the dynamic re-oxygenation mode and the change comparison.

20. An apparatus comprising:
(a) an monitoring device comprising a pulse oximeter;
(b) an oxygen delivery device comprising:
(i) an oxygen delivery output, comprising an oral nasal cannula, and
(ii) an oxygen delivery controller;
(c) a drug delivery device;
(d) a patient server; and
(e) an alarm device;
wherein the oxygen delivery controller is configured to receive a set of patient data from the patient server;
wherein the oxygen delivery controller is configured to determine, based upon the patient data:
(i) an initial delivery concentration,
(ii) a set of transitional delivery concentrations, and
(iii) a change threshold,
wherein the oxygen delivery controller is configured to receive a first set of saturation data from the monitoring device, the first set of saturation data indicating that a patient is experiencing hypoxemia;
wherein the oxygen delivery controller is configured to enter a dynamic re-oxygenation mode in response to receiving the set of saturation data, wherein, upon entering dynamic re-oxygenation mode:
(i) the oxygen delivery controller is configured to deliver oxygen at the initial delivery concentration via the oxygen delivery output, wherein the initial delivery concentration is about 21% oxygen, and
(ii) the drug deliver unit is configured to adjust the rate at which a drug is delivered to the patient;
wherein the oxygen delivery controller is further configured to receive a second set of saturation data from the monitoring device;
wherein the oxygen delivery controller is further configured to perform a transition from the initial delivery concentration to a first transitional delivery concentration of the set of transitional delivery concentrations in response to a change comparison of the second set of saturation data and the first set of saturation data indicating that the patient's oxygen saturation level has improved, wherein each of the set of transitional delivery concentrations is greater than about 21% oxygen; and
wherein the oxygen delivery controller is configured to exit the dynamic re-oxygenation mode, enter a safety mode, and cause the alarm device to activate when the difference between a current saturation level and a previous saturation level exceeds the change threshold.
